(19) United States
(12) Patent Application Publication
(10) Pub. No.: US 2024/0197535 A1
(43) Pub. Date: Jun. 20, 2024

(54) METHOD FOR PROVIDING DEFORMATION-CORRECTED CONTROL DATA FOR A LASER OF A TREATMENT APPARATUS

(71) Applicant: SCHWIND EYE-TECH-SOLUTIONS GMBH, Kleinostheim (DE)

(72) Inventors: Samuel ARBA MOSQUERA, Aschaffenburg (DE); Tobias KEHRER, Kleinostheim (DE)

(21) Appl. No.: 18/539,600

(22) Filed: Dec. 14, 2023

(30) Foreign Application Priority Data
Dec. 16, 2022 (DE) 10 2022 133 647.6

(51) Int. Cl. A61F 9/008

(52) U.S. Cl. CPC A61F 9/00827; A61F 2009/00872

(57) ABSTRACT

The invention relates to a method for providing deformation-corrected control data for a laser (18) of a treatment apparatus (10). The method includes as steps determining (S10) a planned lenticule diameter and a planned refractive power change as correction parameters for correcting a visual disorder of the eye from predetermined examination data; determining deformation-corrected correction parameters for adapting the planned correction parameters, by which a deformation of the cornea (26) is additionally compensated for; wherein either a deformation-corrected lenticule diameter or a deformation-corrected refractive power change is determined from predetermined deformation data as the first deformation-corrected correction parameter (S12) and the respectively other correction parameter is determined as the second deformation-corrected correction parameter by means of the determined first deformation-corrected correction parameter and depending on a mathematical deformation model (S14); and providing the control data for the treatment apparatus (10), which includes the determined deformation-corrected correction parameters.

### FIELD

The present invention relates to a method for providing deformation-corrected control data for a laser of a treatment apparatus for the correction of a cornea of a human or animal eye. In addition, the invention relates to a control device for performing the method, to a treatment apparatus with at least one eye surgical laser and at least one control device, to a computer program, and to a computer-readable medium.

### BACKGROUND

Treatment apparatuses and methods for controlling lasers for correcting an optical visual disorder of a cornea are known in the art. Therein, a pulsed laser and a beam focusing device can for example be formed such that laser beam pulses effect an optical breakthrough in a focus situated within the tissue of the cornea to separate a lenticule from the cornea for correcting the cornea. In the treatment with a treatment apparatus, for example for separating a lenticule, the eye is usually fixed by one or more contact elements of the treatment apparatus. Herein, the contact element is a rigid element, for example a plano-concave lens, which is fitted onto the eye, in particular onto the cornea, in order that the eye is not moved in the treatment. However, it is disadvantageous with such a contact element that a shape of the cornea changes by the contact element, in particular is compressed. Hereby, the shape of the lenticule to be separated can also change, whereby an originally planned treatment can be defective.

After removing the lenticule, the desired correction arises by “collapse” or closure of the cornea. In determining the correction of the cornea, in particular in a refractive power correction, which is performed according to standard methods, however, slight deviations from the actually planned result can occur since an idealized cornea is assumed in closing the cornea.

The above mentioned deformation effects of the cornea, in particular due to the contact element or by not exactly modeled closure of the cornea after removal of the lenticule, can result in undesired deviations of the treatment result upon cumulation of these errors. Therefore, one strives to consider and compensate for these effects in advance, wherein the determination of this compensation is often very complicated and time consuming.

In a treatment with a laser for correcting a visual disorder, a lenticule diameter or a refractive power change is usually planned. If the tissue is deformed, thus, the effect of the deformation acts both in the refractive power change and in the lenticule diameter. Thereby, these correction parameters become dependent on each other. Thus, if one of these correction parameters is deformation-corrected, the respectively other correction parameter also changes, wherein such an adaptation dos not have to be directly proportional and thus is difficult to determine.

### SUMMARY

Therefore, the invention is based on the object to obtain a simplified deformation correction.

This object is solved by the method according to the invention, the devices according to the invention, the computer program according to the invention as well as the computer-readable medium according to the invention. Advantageous embodiments of the invention are specified in the respective dependent claims, wherein advantageous embodiments of the method are to be regarded as advantageous embodiments of the treatment apparatus, of the control device, of the computer program and of the computer-readable medium and vice versa.

An aspect of the invention relates to a method for providing deformation-corrected control data for a laser of a treatment apparatus for the correction of a cornea of a human or animal eye, wherein the method comprises the following steps performed by at least one control device. Therein, an appliance, an appliance component or an appliance group is understood by a control device, which is configured for receiving and evaluating signals as well as for providing, for example generating, control data. For example, the control device can be configured as a control chip, computer program, computer program product or control unit. In the method, determining a planned lenticule diameter and a planned refractive power change as the correction parameters for correcting a visual disorder of the eye from predetermined examination data is effected by the control device. Subsequently, determining deformation-corrected correction parameters for adapting the planned correction parameters, by which a deformation of the cornea is additionally compensated for, is effected, wherein either a deformation-corrected lenticule diameter or a deformation-corrected refractive power change is determined from predetermined deformation data as the first deformation-corrected correction parameter and the respectively other correction parameter is determined as the second deformation-corrected correction parameter by means of the determined first deformation-corrected correction parameter and depending on a mathematical deformation model. Finally, providing the control data to the treatment apparatus, which includes the determined deformation-corrected correction parameters, is effected. Preferably, the treatment apparatus can then be controlled by means of the control data for correcting the cornea of the eye.

In other words, one wishes to know, how the lenticule diameter has to be adapted depending on an adaptation of the refractive power change or the refractive power change has to be adapted depending on the adaptation of the lenticule diameter to compensate for the deformation, which can for example be induced by a contact element or other deformations, in particular closing the cornea after removing the lenticule, or a deformation by a fixing device such as for example a suction ring. Herein, only the effect of the most important correction parameters, that is of the lenticule diameter and the refractive power change, is considered. Therein, a lenticule size in a lateral direction of the cornea is meant by the lenticule diameter, which can in particular include a circular, elliptical or oval contour. For example, the refractive power change can include one or more values, in particular a spherical and/or cylindrical correction, preferably also higher order corrections.

Therein, the required deformation correction can be known for one of these correction parameters, in particular from clinical data and/or simulations. In case of the deformation-corrected refractive power change, the predetermined deformation data can for example be clinical data, in particular from a nomogram or a refractive power difference of topographies before and after a visual disorder correction without deformation correction or a difference of a thickness of the cornea, which can be determined from tomographies, in particular from measurements of a pre/post-treatment, preferably with and without consideration of the epithelium.

In case of the deformation-corrected lenticule diameter, clinical data can in particular be from a nomogram or from a size of an effective disk of the difference of topographies before and after a visual disorder correction without deformation correction or from an effective disk of a thickness difference of tomographies, preferably pre/post-treatment with and without consideration of the epithelium.

After one of these correction parameters and the deformation correction thereof are known, the other one can be calculated based on a deformation model, in which assumptions to the character of the cornea are in particular taken into account. Preferably, a compressibility of the cornea can be taken into account in the deformation model. The mathematical deformation model used thereto can be based on a simulation, an Euler-Bernoulli beam theory, on an assumption of a constant thickness, of a constant volume and/or a low compressibility of the cornea.

Particularly preferably, the deformation model can be based on the Euler-Bernoulli beam theory, by which the cornea, in particular the correction parameters, can be described in the deformed and non-deformed state, wherein the cornea can be described as a volume body in the Euler-Bernoulli beam theory, which is deformed to determin the respective effects on the other correction parameter. A deformation model, which is based on the Euler-Bernoulli beam theory, has proven to be particularly suitable for the simulation of these deformation effects.

The control device, which is provided for determining the control data, can belong to the treatment apparatus or be a control device separate from the treatment apparatus. If the control device is separate from the treatment apparatus, the control data, after its determination, can preferably be subsequently transferred to the treatment apparatus and be stored there. For example, the control data can be determined and provided, respectively, for ablative methods, photodisruptive methods, in particular for a lenticule extraction, cross-linking methods of the cornea (crosslinking) and/or a method for laser-induced refractive index change (LIRIC).

By this aspect of the invention, the advantage arises that a compensation for a deformation of a correction parameter can be provided to another correction parameter, in particular for a transfer of a deformation correction of a lenticule diameter to a refractive power change and vice versa, respectively, to thus easily compensate for deformation effects and thus be able to achieve better results in the treatment. Thus, not only deformations of the cornea, but for example also of a conjunctiva or sclera can in particular be modeled and compensated for.

The invention also includes further embodiments, by which additional advantages are provided.

A further embodiment of the invention provides that a deformation of the cornea, which is generated by a contact element, is compensated for and/or wherein a deformation of the cornea, which is generated in closing the cornea after removal of the lenticule from the cornea, is compensated for. These two deformations represent the most frequent cause of a defective treatment due to deformation effects, wherein the respective correction parameters can be mutually compensated for by means of the deformation model.

A further embodiment of the invention provides that the mathematic deformation model is based on the Euler-Bernoulli beam theory. In other words, the cornea can be described as a volume body, which deforms based on the Euler-Bernoulli beam theory, to describe the deformed cornea. The Euler-Bernoulli beam theory describes an elastic bending of a body, wherein it is assumed that multiple central corneal surfaces are arranged between an anterior corneal surface and a posterior corneal surface, which construct the volume body. According to the Euler-Bernoulli beam theory, one of the central corneal surfaces is a neutral corneal surface or neutral membrane, the surface of which remains constant upon the deformation, wherein the further central corneal surfaces can be described depending on the neutral corneal surface. In modeling the deformation by the contact element, the central corneal surfaces below the neutral corneal surface can for example be compressed and those above are stretched. In modeling the closure of the cornea after removal of the lenticule, the corneal surfaces, which are situated above the lenticule, can be stretched. Based on the Euler-Bernoulli beam theory, it can be mathematically calculated how the central corneal surfaces change upon an elastic deformation, in particular in relation to the neutral corneal surface. The use of the Euler-Bernoulli beam theory as the deformation model has proven to be particularly suitable since it particularly accurately describes the deformation of the cornea. Thus, improved corrections for a deformation can also be modeled.

A further embodiment provides that the second deformation-corrected correction parameter is determined in that the equation s_{D}*(sₓ*s_{y})^{(-v/2)}=1 is satisfied, wherein sp is a ratio of the deformation-corrected refractive power change to the planned refractive power change, sₓ and s_{y} are the ratio of the deformation-corrected lenticule diameter to the planned lenticule diameter in x- and y-direction and v is a deformation parameter of the cornea, which is determined from the deformation model. In other words, the indicated equation describes a correlation of the refractive power change and the lenticule diameter, wherein they correlate with a deformation parameter or deformation exponent v, by which a compressibility of the cornea can be provided. In the indicated equation, two values are preferably known from s_{D}, xₓ or s_{y} and the respectively lacking one is determined.

Preferably, it is provided that sₓ and s_{y} are the same. Thus, the above shown equation results in s_{D}*(s_{x/y})^{-v}=1 wherein one value is then known and the other one is determined.

Particularly preferably, it is provided that the deformation factor v is a value in a range from −2 to −4, in particular −2, −3, −8^{1/2} or −4. These values for the deformation factor are particularly preferred values, which can be determined from the deformation model. Herein, it was recognized that the value −2 preserves a central/maximum thickness, in particular for an assumed parabolic profile. Thus, residual tissue can be preserved. For the value −4, it is assumed that a volume remains, in particular for approximately parabolic profiles, wherein an effect can thus be maximized. By the average value −3 or the geometric mean from the (negative) root from 8, a balancing compromise between preservation of a central thickness and a volume can advantageously be obtained.

Particularly advantageously, it is provided that the value of the deformation parameter v is determined based on a statistical evaluation of preceding treatments, in particular based on comparable already performed treatments. This means that preceding treatments can be classified or grouped, wherein they are each analyzed with the deformation model. Thus, it can be statistically determined, which deformation factor in comparable treatments can be assumed for a planned further treatment.

A second aspect of the present invention relates to a control device, which is configured to perform the above described method. The above listed advantages arise. The control device can for example be configured as a control chip, control unit or application program (“app”). Preferably, the control device can comprise a processor device and/or a data memory. An appliance or an appliance component for electronic data processing is understood by a processor device. For example, the processor device can comprise at least one microcontroller and/or at least one microprocessor. Preferably, a program code for performing the method according to the invention can be stored on the optional data memory. The program code can then be adapted, upon execution by the processor device, to cause the control device to perform one of the above described embodiments of the method according to the invention.

A third aspect of the present invention relates to a treatment apparatus with at least one eye surgical laser for the separation of a lenticule with predefined interfaces from a human or animal eye by means of optical breakthroughs and/or ablation, and at least one control device for the laser or lasers, which is formed to execute the steps of the method according to the first aspect of the invention.

In a further advantageous embodiment of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous embodiments of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

Further features and the advantages thereof can be taken from the descriptions of the first inventive aspect, wherein advantageous embodiments of each inventive aspect are to be regarded as advantageous embodiments of the respectively other inventive aspect.

A fourth aspect of the invention relates to a computer program including commands, which cause the control device according to the second inventive aspect to execute the method steps according to the first inventive aspect.

A fifth aspect of the invention relates to a computer-readable medium, on which the computer program according to the fourth inventive aspect is stored. Further features and the advantages thereof can be taken from the descriptions of the first to fourth inventive aspects, wherein advantageous embodiments of each inventive aspect are to be regarded as advantageous embodiments of the respectively other inventive aspect.

### BRIEF DESCRIPTION OF THE DRAWINGS

Further features of the invention are apparent from the claims, the figures and the description of the figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separate feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 depicts a schematic representation of a treatment apparatus according to an exemplary embodiment.

FIG. 2 depicts a schematic method diagram for providing deformation-corrected control data according to an exemplary embodiment.

FIG. 3a depicts a schematically illustrated cornea of the deformation model in the non-deformed state.

FIG. 3b depicts the cornea of the deformation model deformed by a contact element.

FIG. 4a depicts a schematically illustrated cornea of the deformation model in the non-deformed state before removal of a lenticule.

FIG. 4b depicts the deformed cornea of the cornea deformation model after closing the lenticule.

FIG. 5 depicts a double-logarithmic representation of the relation between the lenticule diameter and the refractive power change determined with the deformation model.

In the figures, identical or functionally identical elements are provided with the same reference characters.

### DETAILED DESCRIPTION

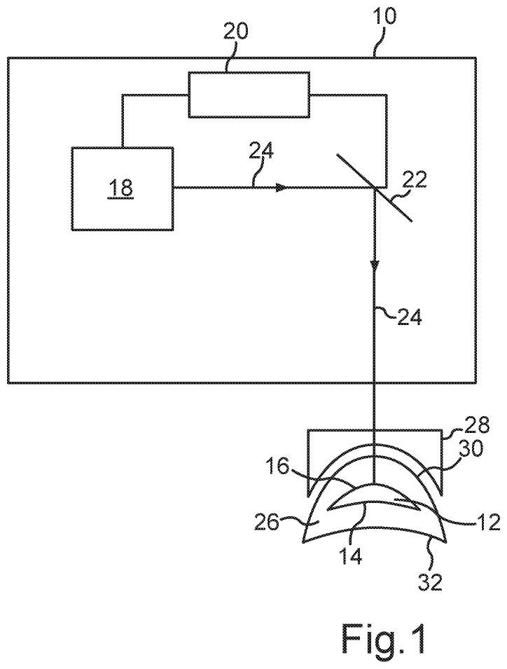

FIG. 1 shows a schematic representation of a treatment apparatus 10 with an eye surgical laser 18 for the separation of a lenticule 12 defined by control data from a cornea 26 by means of photodisruption and/or ablation, wherein the cornea 26 is delimited in the direction of an optical axis by an anterior corneal surface 30 and a posterior corneal surface 32. For separating the lenticule 12, a posterior interface 14 and an anterior interface 16 of the lenticule 12 are preset in the control data, on which a cavitation bubble path for separating the lenticule 12 from the cornea 26 can be generated. One recognizes that a control device 20 for the laser 18 can be formed besides the laser 18 such that it can emit pulsed laser pulses for example in a predefined pattern for generating the interfaces 14, 16. Alternatively, the control device 20 can be a control device 20 external with respect to the treatment apparatus 10.

Furthermore, the FIG. 1 shows that the laser beam 24 generated by the laser 18 is deflected towards the cornea 26 by means of a beam device 22, namely a beam deflection device such as for example a rotation scanner. The beam deflection device 22 is also controlled by the control device 20 to generate the interfaces 14, 16, preferably also incisions or cuts, along preset incision progressions.

Preferably, the laser 18 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. In addition, the control device 20 optionally comprises a storage device (not illustrated) for at least temporary storage of at least one control dataset, wherein the control dataset or datasets include(s) control data for positioning and/or for focusing individual laser pulses in the cornea. The position data and/or the focusing data of the individual laser pulses, that is the lenticule geometry of the lenticule 12 to be separated, are generated based on predetermined control data, in particular from a previously measured topography and/or pachymetry and/or the morphology of the cornea or of the optical visual disorder correction to be generated.

For determining the visual disorder data, which can for example indicate a value in diopters, suitable examination data for describing the visual disorder can be received by the control device 20 from a data server or the examination data can be directly input into the control device 20.

Further, a contact element 28 can be provided, which can belong to the treatment apparatus 10. Alternatively, the contact element 28 can also be provided separately from the treatment apparatus 10. The contact element 28, which can also be referred to as patient interface or fixing system, serves for fixing the eye and the cornea 26, respectively, for the treatment. Hereto, the contact element 28 can comprise a plano-concave lens, which is adapted to the cornea 26 for fixing. However, by fixing by means of the contact element 28, it can occur that the cornea 26 deforms and thus the geometry of the lenticule 12 does no longer have the originally planned dimensions. Therefore, it can occur that a planned refractive power value or refractive power value to be corrected for example deviates from an achieved refractive power value after the treatment with the treatment apparatus 10.

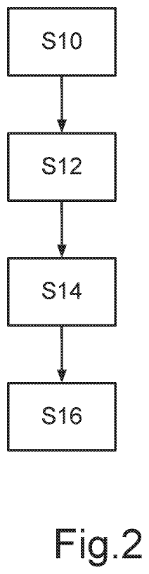

Therefore, the deformations, which are induced by the contact element 28 as well as by closing the cornea 26 after removing the lenticule 12, are preferably to be taken into account in the treatment planning. In particular, the planned lenticule diameter and the planned refractive power change are essential correction parameters for correcting the visual disorder of the cornea 26, wherein these correction parameters depend on each other, such as for example described in the Munnerlyn formula. Herein, methods are known to provide a deformation correction for one of these correction parameters, for example from clinical data or simulations. However, it has become apparent that the respectively other correction parameter cannot be correspondingly adapted with the known methods since there is apparently a non-linear correlation in the deformation correction of these correction parameters. For considering an improved deformation correction, therefore, it is provided to perform the method schematically illustrated in FIG. 2, which can for example be performed by the control device 20.

In a step S10, the lenticule diameter and the refractive power change are planned from predetermined examination data. The planned lenticule diameter and the planned refractive power change correspond to the not deformation-corrected correction parameters as they can for example be used for the determination of a geometry of the lenticule by means of the Munnerlyn formula.

In a step S12, one of these correction parameters is deformation-corrected by means of predetermined deformation data. This means that either the lenticule diameter or the refractive power change is adapted such that a deformation to be expected, for example a deformation by the contact element 28, is compensated for. This can be performed according to known methods, for example based on statistical data (deformation data).

In order to correct the other correction parameter, which was not adapted in step S12, for the deformation, it can subsequently be determined in a step S14 with the aid of a mathematical model. In particular, a correlation between the first correction parameter, which was already deformation-corrected, and the second correction parameter, which is still to be deformation-corrected, can in particular be established by the mathematical deformation model. It has become apparent that this correlation can be particularly preferably represented with the Euler-Bernoulli beam theory.

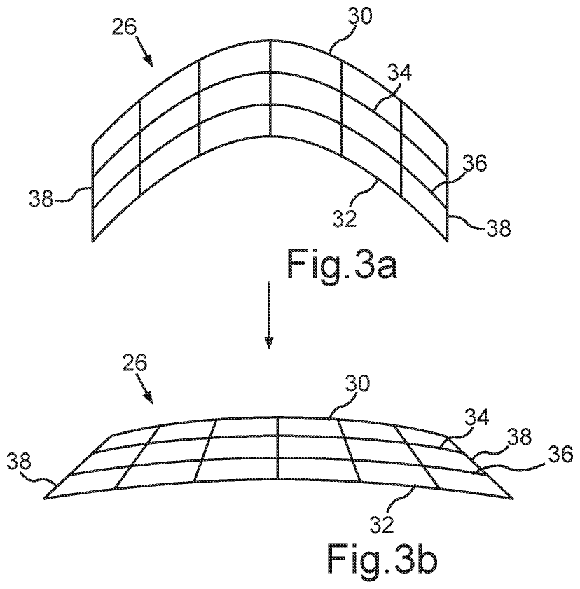

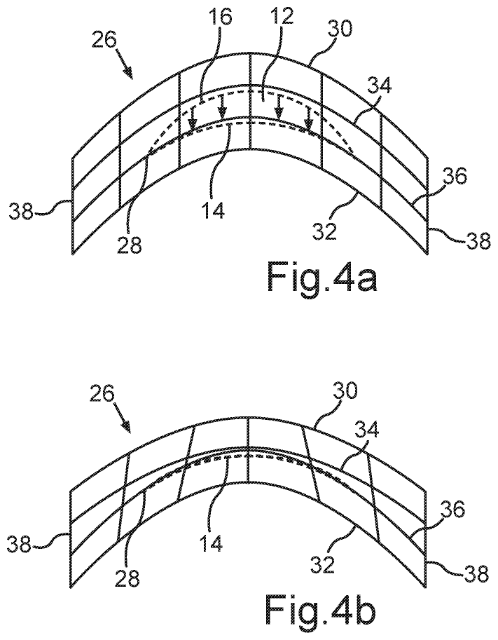

For exemplifying the Euler-Bernoulli beam theory, the deformation of the volume body of the cornea 26 is shown for the deformation by the contact element 28 in FIGS. 3a and 3b and for the deformation, which occurs in closing the cornea 26 after removing the lenticule 12 in FIGS. 4a and 4b.

Herein, FIG. 3a for example shows the volume body of the cornea 26 in a free state before the deformation by the contact element 28, which is not illustrated in this figure. Therein, the volume body can be delimited in the direction of the optical axis by the anterior corneal surface 30 and the posterior corneal surface 32 and in radial direction (laterally) by lateral interfaces 38. Herein, the anterior corneal surface 30 and the posterior corneal surface 32 can be provided as ellipsoids, wherein a two-dimensional cross-section through the volume body is shown in this figure for exemplification, and the volume body can be present in a three-dimensional form, in particular rotation-symmetric. Besides the anterior and posterior corneal surfaces 30, 32, central corneal surfaces 34, 36 of the volume body are also illustrated, wherein a central corneal surface can be provided for each position in z-direction (direction of the optical axis) within the volume body, which is not shown here for reasons of clarity. One of the central corneal surfaces, for example the central corneal surface 36, can be a neutral corneal surface or neutral membrane, which has the same surface before and after the deformation according to the Euler-Bernoulli beam theory, which is taken into account in modeling the cornea 26 based on the cornea deformation model. Preferably, a respectively central corneal surface 34 can be described in relation to this neutral corneal surface in the cornea deformation model.

Thus, a radius of curvature of a respectively central corneal surface 34 can preferably be described by means of the cornea deformation model according to the formula

$$\frac{1}{{r}_{cent , pre}} = ( \frac{q}{{r}_{ca}} + \frac{1 - q}{{r}_{cp}} )$$

wherein it provides the radius of curvature of the central corneal surface 34 before the deformation (r_{cent,pre}). Therein, r_{ca} describes the radius of curvature of the anterior corneal surface 30 and r_{cp} describes the radius of curvature of the posterior corneal surface 32. The variable q describes a relative position of the central corneal surface 34 to the neutral corneal surface 36, wherein q can take a value between 0 and 1.

In similar manner, a position in z-direction, which is dependent on the radial position, can also be described to the radius of curvature, wherein the z-direction extends in the direction of the optical axis. It can be described for the respective central corneal surface 34 with the formula

$${z}_{cent , pre} ( {τ}_{x} ) = ( q - 1 ) {d}_{cc} - \frac{{r}_{x}^{2}}{2} ( \frac{q}{{r}_{ca}} + \frac{1 - q}{{r}_{cp}} )$$

wherein r_{X} describes a radial position starting from the center of the cornea 26 and d_{CC} describes a central thickness of the cornea 26 at the highest point or inflection point of the cornea 26.

In the deformation of the cornea 26 by the contact element 28, it can be provided in the deformation model that the radius of curvature of the anterior corneal surface 30 is adapted to a radius of curvature of the contact element 28. This situation is for example illustrated in FIG. 3b, wherein the contact element 28 is not shown here for reasons of clarity. It is seen that the anterior corneal surface 30 is impressed and thus also the central corneal surfaces 34 and 36. However, according to the Euler-Bernoulli beam theory, it remains further considered herein that the neutral corneal surface 36 has the same surface as before the deformation. In this deformation, it is assumed that the volume body can freely deform and is not delimited towards the sides.

In FIG. 4a, the cornea 26 is illustrated in a non-deformed state before the removal of the lenticule 12. Here too, the cornea 26 can be modeled as a volume body, which is formed of respective central corneal surfaces 34, 36, wherein for determining the deformed cornea in the deformation model, the anterior interface 16 of the lenticule is pressed onto the posterior interface 14 of the lenticule 12, whereby the curvatures of the corneal surfaces 30, 34 located above change. Therein, the deformation model is based on the same principles and formulas as already described to the FIGS. 3a and 3b.

In the deformation of the cornea 26 by closing the area of the lenticule 12, it can be provided in the deformation model that the radius of curvature of the anterior interface 16 is adapted to a radius of curvature of the anterior interface 14, such that the cornea 26 according to FIG. 4b results. Herein, the anterior interface 16 can move downwards onto the posterior interface 14, whereby the corneal surfaces located above the anterior interface are thus also adapted, in particular the neutral corneal surface 34 and the anterior corneal surface 30.

Now, in order to establish a correlation between the above mentioned correction parameters, thus between the lenticule diameter and the refractive power change, with this deformation model, which is preferably based on the Euler-Bernoulli beam theory, the volume body of the cornea 26 modeled by means of the deformation model can be deformed, wherein an effect of the deformation of a correction parameter on the other one is examined.

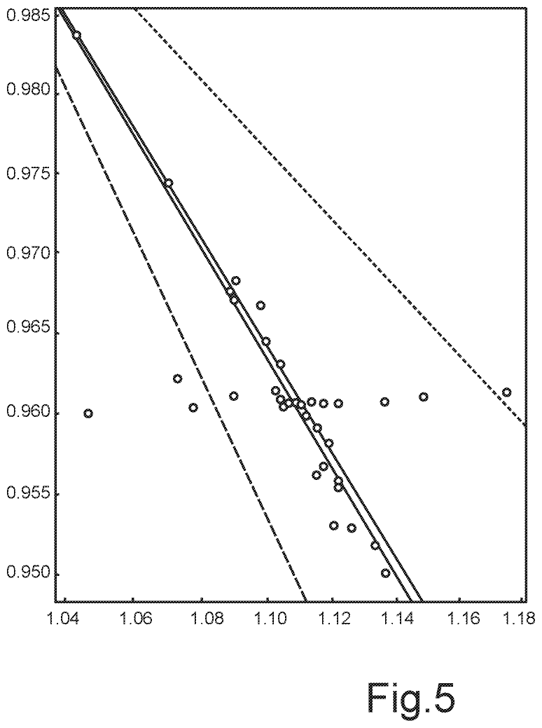

For example, this is shown in FIG. 5, in which a double-logarithmic representation of the relation between the lenticule diameter and the refractive power change determined with the deformation model is provided. Therein, the ratio of the deformation-corrected lenticule diameter to the uncorrected or planned lenticule diameter is plotted on the y-axis, and the ratio of the corrected refractive power change to the uncorrected or planned refractive power change is plotted on the x-axis.

Therein, the points shown in the diagram of FIG. 5 correspond to different corneal parameters, which have been assumed for examining the correlation. This means, different corneal parameters were changed to obtain the effect on the correlation of the lenticule diameter and the refractive power change upon the deformation correction. The changed or examined corneal parameters for example include a radius of curvature of an anterior corneal surface and/or an optical distance between the anterior corneal surface and a posterior corneal surface and/or a thickness of the cornea and/or a radial distance from a limbus to a center of the cornea and/or an optical distance between the anterior corneal surface and an anterior interface of a lenticule to be separated and/or a radius of the anterior interface of the lenticule to be separated and/or a thickness of the lenticule and/or a radius of curvature of a contact element and/or a relative thickness of the cornea and/or an incision angle of an incision cut.

These scenarios examined by means of the deformation model can subsequently be fitted to provide the correlation of the lenticule diameter and the refractive power change for the deformation correction, wherein the solid lines represent the fit curves (in a fit curve (left solid line), scenarios with corneal parameters were removed, which do almost not influence the scaling of the lenticule 12 in the x-y plane). Furthermore, the dashed line (in the diagram on the left) shows the correlation upon assumption of a preserved volume of the cornea 26 and the dotted line (in the diagram on the right) of a preserved thickness of the cornea 26.

Thus, as the correlation between the lenticule diameter and the refractive power change, the equation s_{D}*(sₓ*s_{y})^{(-v/2)}=1 can be obtained from the fit curves, which is to be satisfied, wherein s_{D} is a ratio of the deformation-corrected refractive power change to the planned refractive power change, sₓ and s_{y} are the ratio of the deformation-corrected lenticule diameter to the planned lenticule diameter in x- and y-direction and v is a deformation parameter of the cornea. Preferably, the deformation factor v is a value in a range from −2 to −4, in particular −2, −3, −8^{(1/2)} or −4. Particularly preferably, the value of the deformation parameter v for an individual patient can also be determined based on a statistical evaluation of preceding treatments, in particular based on comparable already performed treatments.

After determining the second deformation-corrected correction parameter according to the above shown principle, control data can finally be provided for the treatment apparatus 10 in a step S16, which includes the determined deformation-corrected correction parameters. Preferably, the control device 20 can subsequently control the laser 18 and/or the beam deflection device 22 by means of the control data for correcting the visual disorder of the cornea 26.

1. A method for providing deformation-corrected control data for a laser of a treatment apparatus for correcting a cornea of a human or animal eye, wherein the method comprises the following steps performed by at least one control device:
determining a planned lenticule diameter and a planned refractive power change as correction parameters for correcting a visual disorder of the human or animal eye from predetermined examination data;
determining deformation-corrected correction parameters for adapting the correction parameters, by which a deformation of the cornea is additionally compensated for;
wherein either a deformation-corrected lenticule diameter or a deformation-corrected refractive power change is determined from predetermined deformation data as a first deformation-corrected correction parameter, and
a respective other of the correction parameters is determined as a second deformation-corrected correction parameter by means of the determined first deformation-corrected correction parameter and depending on a mathematical deformation model; and
providing the deformation-corrected control data for the treatment apparatus, which includes the determined first and second deformation-corrected correction parameters.

2. The method according to claim 1, wherein a deformation of the cornea, which is generated by a contact element, is compensated for and/or wherein a deformation of the cornea, which is generated in closing the cornea after removing a lenticule from the cornea, is compensated for.

3. The method according to claim 1, wherein the mathematical deformation model is based on the Euler-Bernoulli beam theory.

4. The method according to claim 1, wherein the second deformation-corrected correction parameter is determined in that an equation s_{D}*(sₓ*s_{y})^{(-v/2)}=1 is satisfied, wherein sp is a ratio of the deformation-corrected refractive power change to the planned refractive power change, sₓ and s_{y} are the ratio of the deformation-corrected lenticule diameter to the planned lenticule diameter in x- and y-direction and v is a deformation parameter of the cornea, which is determined from the deformation model.

5. The method according to claim 4, wherein sₓ and s_{y} are the same.

6. The method according to claim 4, wherein a deformation factor v is a value in a range from −2 to −4, in particular −2, −3, −8^{(1/2)} or −4.

7. The method according to claim 4, wherein a value of the deformation parameter v is determined based on a statistical evaluation of preceding treatments, in particular based on comparable already performed treatments.

8. A control device, which is configured to perform a method according to claim 1.

9. A treatment apparatus with at least one eye surgical laser for separation of a lenticule with predefined interfaces from a human or animal eye by cavitation bubbles, and at least one control device according to claim 8.

10. The treatment apparatus according to claim 9, wherein the laser is suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kHz, preferably between 100 kHz and 100 MHz.

11. The treatment apparatus according to claim 9, wherein the control device comprises at least one storage device for at least temporary storage of at least one control dataset, wherein the at least one control dataset includes control data for positioning and/or for focusing individual laser pulses in the cornea; and
the treatment apparatus includes at least one beam device for beam guidance and/or beam shaping and/or beam deflection and/or beam focusing of a laser beam of the laser.

12. A non-transitory computer readable medium configured for storing a computer program, the computer program including commands which cause a control device to execute the method steps according to claim 1.

13. (canceled)
